(19) United States
(12) Patent Application Publication
(10) Pub. No.: US 2010/0120899 A1
(43) Pub. Date: May 13, 2010

(54) Tumor Repressor Genes in the Prevention of Cancer

(76) Inventors: Roger H. Reeves, Silver Spring, MD (US); Thomas E. Sussan, Baltimore, MD (US)

Correspondence Address: DLA PIPER LLP (US), 4365 EXECUTIVE DRIVE, SUITE 1100, SAN DIEGO, CA, 92121-2133, US

(21) Appl. No.: 12/523,155

(22) PCT Filed: Jan. 16, 2008
(86) PCT No.: PCT/US08/51216

§ 371 Date: Jan. 15, 2010

Related U.S. Application Data
(60) Provisional application No. 60/880,654, filed on Jan. 16, 2007.
(60) Provisional application No. 61/005,594, filed on Dec. 5, 2007.

(51) Int. Cl. A61K 31/7052; C12Q 1/68; A61P 35/00

(52) U.S. Cl. 514/44 R; 435/6

(57) ABSTRACT

The present invention provides methods for the use of tumor repressor molecules as a prognostic screen for cancer, risk assessment, prognosis, treatment and prevention of cancer in a subject. Furthermore, this invention provides a method to identify new genes that are tumor repressors.

### FIELD OF THE INVENTION

The present invention relates generally to proliferative diseases such as cancer and, more specifically to the use of tumor repressor molecules that can be used as a prognostic screen for cancer, risk assessment, prognosis, treatment and prevention of cancer.

### BACKGROUND INFORMATION

Cancer is one of the leading causes of death in the United States. Each year, more than half a million Americans die from cancer, and more than one million are newly diagnosed with the disease. Cancerous tumors result when a cell escapes from its normal growth regulatory mechanisms and proliferates in an uncontrolled fashion. Tumor cells can metastasize to secondary sites if treatment of the primary tumor is either not complete or not initiated before substantial progression of the disease. Early diagnosis and treatment of tumors is therefore essential for survival.

The exact cause of cancer is not known, but links between certain activities such as smoking or exposure to carcinogens and certain inherited factors, and the incidence of certain types of cancers and tumors has been shown by a number of researchers. Cancer involves the clonal replication of populations of cells that have gained competitive advantage over normal cells through the alteration of regulatory genes. Regulatory genes can be broadly classified into “oncogenes” which, when activated or overexpressed, promote unregulated cell proliferation, and “tumor suppressor genes” which, when inactivated or underexpressed fail to prevent abnormal cell proliferation. Oncogenes promote tumor formation when mutated; a normal copy of a tumor suppressor gene is required to prevent transformation. Loss of function or inactivation of tumor suppressor genes is thought to play a central role in the initiation and progression of a significant number of human cancers.

Many types of chemotherapeutic agents have been shown to be effective against cancers and tumor cells, but not all types of cancers and tumors respond to these agents. Unfortunately, many of these agents are toxic and also destroy normal cells. The exact mechanism for the action of these chemotherapeutic agents are not always known.

Despite advances in the field of cancer treatment, the leading therapies to date are surgery, radiation and chemotherapy. Chemotherapeutic approaches are often used for cancers that are metastasized or ones that are particularly aggressive. Such cytocidal or cytostatic agents work best on cancers whose cells are rapidly dividing. To date, hormones, in particular estrogen, progesterone and testosterone, and some antibiotics produced by a variety of microbes, alkylating agents, and anti-metabolites form the bulk of therapies available to oncologists. Ideally cytotoxic agents that have specificity for cancer and tumor cells while not affecting normal cells would be extremely desirable. Unfortunately, none have been found and instead agents which target especially rapidly dividing cells (both tumor and normal) have been used.

Down syndrome (DS), which is caused by trisomy for human chromosome 21 (Hsa21), is the most common genetic cause of mental retardation. DS occurs in 1 in 800-1000 live births and results in over 80 different clinical phenotypes, including craniofacial abnormalities, a small hypocellular brain with a disproportionately small cerebellum, Alzheimer-like histopathology, and an elevated risk for congenital heart defects, Hirschsprung's disease, and leukemia.

DS is associated with two contrary cancer-related phenotypes. The first observation of a patient with leukemia and DS was made in 1930, and an increased risk of leukemia among individuals with DS was established by 1955. Acute megakaryoblastic leukemia (AMKL) occurs approximately 500-fold more frequently in individuals with DS than in the general population. AMKL almost always occurs in concert with a somatic mutation in the GATAI transcription factor.

Epidemiological studies indicate that the overall risk of solid tumors is significantly lower among individuals with DS than in the general population. However, the degree to which DS is observed to reduce tumor incidence varies by more than an order of magnitude among these studies, and the variability becomes even greater when the cancer risks are assessed for individual tissues, since even large studies include relatively few individuals with the same types of tumors. (Table 1).

TABLE 1
Summary of epidemiological evidence for tumor repression in DS.

| | Author | | | | | | | | |
|---|---|---|---|---|---|---|---|---|---|
| | Hill | Hasle | Hi!l | Yang | Satge | Scholl | Boker | Hermon | Goldacre |
| | (Hill, | (Hasle, | (Hill, | (Yang, | (Satge, | (Scholl, | (Boker, | (Hermon, | (Goldacre, |
| | Gridley | Clemmensen | Gridley | Rasmussen | Sommelet | Stein | Blumstein | Alberman | Wotton |
| | et al. 2003) | et al. 2000) | et al. 2003) | et al. 2002) | et al. 1998) | et al. 1982) | et al. 2001) | et al. 2001) | et al. 2004) |
| Incidence/Mortality | Incidence | Incidence | Mortality | Mortality | Incidence | Mortality | Incidence | Mortality | Incidence |
| Study | | | | | | | | | |
| All Solid Tumors | 0.8 | 0.5 | | 0.07 | Reduced | | | | 1.2 |
| Gastric | | | | | | 0.32 | 11.9 | | |
| Stomach | 3.5 | 1.1 | 6.4 | 0.13 | Reduced | | | 1.53 | |
| Small Intestine | 8.3 | | 3.3 | | Reduced | | | | |
| Colon | 2.1 | 0.89 | 7.2 | 0.08 | Reduced | | | | 3.1 |
| Peritoneum | | 67.77 | | | | | | | |
| Lung | | 0.24 | | 0.2 | Reduced | 0 | | | |
| Liver | 6 | | | 0.41 | | | | | |
| Breast | 0.5 | | | 0.04 | Reduced | 0.09 | | 0.62 | |
| Endometrial | 2.2 | | | 0.22 | Reduced | | | | |
| (Uterus) | | | | | | | | | |
| Ovary | | 1.97 | | 0.7 | | | | 4.05 | |
| Testis | 3.7 | 1.86 | 25.2 | 3.23 | Increased | | | 8.4 | 12 |
| Other Male Genital | 45.5 | | | | Increased | 0.11 | | | |
| Prostate | | | | 0.08 | | | | | |
| Kidney | 0.6 | 0.84 | | 0.08 | Reduced | | | | |
| Bladder | | 1.69 | | 0.2 | | | | | |
| Skin | | 0.25 | | 0.06 | | | | | |
| Brain | 0.7 | 0.3 | | 0.09 | Reduced | | | | |
| Eye | | 3.68 | | | Increased | | | | |
| Oral | | | | 0.65 | Reduced | | | | |
| Pancreas | | | 1.4 | 0.14 | Increased | | | | |
| Gall Bladder | | | 8.2 | | | | | | |
| Bone | | | | | Increased | | | | |
| Endocrine | 1.4 | | | | | | | | |
| Unspecified | 0.6 | 3.27 | 0.6 | | | 0.13 | | | |
| Total Tumors | 28 | 24 | 20 | 217 | | 10 | 2 | 5 | 5 |
| Observed | | | | | | | | | |

Note:
Numbers correspond to relative frequency of DS tumors compared to expected frequency. Only qualitative data are presented.

Improvements in healthcare over the last 20 years have greatly increased life expectancy in DS, thereby increasing the lifetime window for developing cancer. The degree to which DS is observed to reduce tumor incidence in specific cancers relies on relatively small subsets of individuals with imprecisely defined disease and frequencies predicted across studies vary by more than an order of magnitude. These retrospective analyses of hospital and death records do not provide insight into whether an altered tumor profile in DS is the result of genetic or environmental conditions. An epidemiological approach cannot define the genetic mechanisms by which gene dosage reduces tumor formation. Conflicting results about a protective effect of trisomy 21 continue to be reported.

Mouse models of DS and of cancer provide a more direct approach to address whether and how trisomy protects against solid tumors. Several genetic mouse models of DS exist. The most widely-used of these models is the Ts65Dn mouse, which is trisomic for orthologs of approximately half of the 261 protein coding genes on Hsa21. (FIG. 1) This mouse recapitulates in detail several phenotypes of DS, including impairments in learning and memory degeneration of basal forebrain cholinergic neurons with aging, small cerebellum, fewer granule cell neurons and reduced cell proliferation in the dentate gyrus, and dysmorphology of the craniofacial skeleton, mandible and cranial vault. The Ts1Rlr mouse has segmental trisomy for a subset of the genes represented in Ts65Dn which correspond to a “critical region” on Hsa21 which harbors genes sufficient to cause a number of DS phenotypes. (Table 2).

TABLE 2
Genes conserved with Hsa21 that are triplicated
in Ts65Dn mouse and monosomic in Ms1Rhr¹.

| Symbol | Status¹ | Gene name |
|---|---|---|
| CBR3 | C | Carbonyl reductase |
| C21orf5 | C | |
| AK009785 | MC | |
| K1AA0136 | C | ATP-binding domains |
| CHAF1B | C | Chromatin assembly factor |
| CLDN14 | C | Cell adhesion protein in tight junctions |
| SIM2 | C | Transcription factor; HLH, 2 PAS, 1 PAC domain |
| HLCS | C | Holocarboxylase synthase |
| DSCR6 | MC | |
| DSCR5 | C | 2 transmembrane domains; Down syndrome |
| | | critical region protein 5 |
| TTC3 | C | Tetratricopeptide repeats |
| DSCR3 | C | Vacuolar protein sorting-associated protein |
| | | (Vps) 26 motif |
| DYRK1A | C | serine-threonine protein kinase; tyrosine |
| | | phosphorylation regulated |
| as-DYRK1 | C | |
| KCNJ6 | C | Potassium inwardly-rectifying channel, |
| | | subfamily J, member 6 |
| KCNJ15 | C | Potassium inwardly-rectifying channel, |
| | | subfamily J, member 15 |
| as-KCNJ15 | MC | |
| ERG | C | ETS-related; SAM/Pointed and ETS domains; |
| | | transcription factor |
| ETS2 | C | v-ets erythroblastosis virus E26 oncogene |
| | | homolog 2 (avian); transcription factor |
| DSCR2 | C | Leucine rich |
| WDR9 | C | 8 Trp-Aps domains; 2 bromo (DNA binding) |
| | | domains |
| HMG14 | C | high-mobility group (nonhistone chromosomal) |
| | | protein 14 |
| WRB | C | Signal sequence; 2 transmembrane domains; |
| | | trp-rich C terminus |
| C21orf13 | C | |
| SH3BGR | C | Signal sequence; Pro-rich putative SH3 |
| | | domain; Glu-rich C-terminus |
| B3GALT5 | C | UDP-Gal: betaGlcNAc beta 1,3-galacto- |
| | | syltransferase, polypeptide 5 |
| IGSF5 | C | immunoglobulin superfamily, member 5 |
| PCP4 | C | PEP19; brain specific peptide |
| DSCAM | C | Down syndrome ceil adhesion molecule |
| as-DSCAM | MC | |
| BACE2 | C | Asparty protease; b-site APP cleavage |
| MX1 | C | Interferon-induced cellular resistance |
| | | mediator protein; Dynamin and Dynamin |
| | | GTPase effector domains |
| C21orf11 | C | |

¹C, conserved, MC, moderately conserved

The laboratory mouse also been used to study a variety of different cancers, including some in which tumor burden is quantifiable. The Apc^{Min} mouse model has been widely studied in this regard. Mice that are heterozygous for this mutation (herein, Apc^{Min} mice) accumulate tumors analogous to those in familial adenomatous polyposis (FAP) along the wall of the small intestine and colon. APC is also mutated in a high proportion of spontaneous intestinal cancers in human beings. While the Apc^{Min} mutation is completely penetrant, the number of tumors that develop is dependent on both genetic modifier genes and environmental factors. Several modifier genes affecting tumor number in Apc^{Min} mice have been mapped, and of these, modifier of Min-1 (Mom1) has been cloned and shown to encode the phospholipase A2 gene (Pla2g2A). Some inbred strains, including C57BL/6, carry a mutation in this gene (Pla2g2A^{Mom l-s}, herein Momls) that increases tumor number in mice that also carry the Apc^{Min} mutation; the wild type allele (Pla2g2A^{mom l-r}, herein Momlr) reduces tumor number. Mice carrying the Apc^{Min} mutation that are homozygous for Momls develop about twice as many tumors as mice that are heterozygous at this locus, while homozygous wild type (Momlr/Momlr) mice have about half as many tumors as heterozygotes.

One gene located on Hsa21 is Ets2. Ets2 encodes a phosphorylated transcription factor, which has been shown to act as both a proto-oncogene and a putative tumor suppressor. Over-expression of Ets2 has been linked to hepatocarcinoma, esophageal squamous cell carcinoma, prostate, and breast cancers. However, its expression has also been shown to induce p53 and caspase 3-dependent apoptosis, activate Ras-induced senescence, enhance p19 (INK4a) expression, and activate a Moml-related gene, phospholipase A2 activating protein.

Eleven genes from Hsa21 have also been implicated in the mitogen-activated protein kinase (MAPK) pathway. The MAPK pathway, in response to a variety of extracellular stimuli, induces proliferation, differentiation, development, inflammation, or apoptosis. As a result, over-expression of this pathway is a key component of tumor growth and invasion. Ts65Dn mice, which are trisomic for 9 of these genes, have a 40-45% reduction in phosphorylated Erk1 and Erk2 levels, indicating that MAPK signaling is perturbed in trisomic mice.

Several extracellular matrix proteins are encoded on Hsa21. Studies have demonstrated that the extracellular matrix is capable of modulating tumor growth. These studies have shown that exogenous cancer cells form tumors less efficiently (fewer tumors, slower growth) in breast tissue expressing lower levels of Ets2.

The development or identification of drugs or agents that would diagnose, treat and prevent cancer would be a breakthrough. The identification and use of tumor repressors in diagnosis, treatment and prevention of cancer has not yet been described. Thus, a need exists for such a method. The present invention describes the use of tumor repressors found on Hsa21 for diagnosis of cancer, treatment of cancer, prevention of cancer, and identification of tumor repressor genes. Tumor repressors found on Hsa21 are protective against tumors, as demonstrated by a significantly reduced tumor burden in Ts65Dn, Apc^{Min} mice. Ts65Dn mice have a baseline of higher apoptosis in intestinal crypts and increased antioxidant levels compared to euploid, suggesting specific ways in which aneuploidy might result in this protective effect.

### SUMMARY OF THE INVENTION

The present invention is based on the finding that genes located on chromosome 21 can decrease the incidence of cancer. This seminal discovery is useful for cancer screening, risk-assessment, prognosis, treatment, and prevention. Furthermore, this discovery is useful for the identification of new genes that are tumor repressors.

In one embodiment, there are provided methods for determining whether a subject has or is at risk of having cancer by measuring the level of expression of a polynucleotide encoding a tumor repressor or the activity of the tumor repressor in a sample from the subject, wherein a decreased level relative to a reference level is indicative of a subject that has or is at risk of having cancer. In one aspect, the polynucleotide encoding the tumor repressor or the activity of the tumor repressor is at least one polynucleotide located on human chromosome 21. In another aspect, the polynucleotide encoding the tumor repressor or the activity of the tumor repressor is at least one polynucleotide located on the long arm of human chromosome 21. In yet another aspect, the polynucleotide encoding the tumor repressor or the activity of the tumor repressor is at least one polynucleotide located on region 21q21-21q22.13 of human chromosome 21. In another aspect, the polynucleotide encoding the tumor repressor or the activity of the tumor repressor is one or more polynucleotides located on region 21q21-21q22.13 of human chromosome 21. In another aspect, the polynucleotide encoding the tumor repressor or the activity of the tumor repressor is selected from at least one of the genes indicated in Table 2. In another aspect, the polynucleotide encoding the tumor repressor or the activity of the tumor repressor is the Ets2 polynucleotide. In yet another aspect, the polynucleotide encoding the tumor repressor or the activity of the tumor repressor further contains about 500,000 nucleotides on either side of the Ets2 polynucleotide.

In one embodiment of the present invention, a method of treating cancer in a subject is provided by administering to the subject in need thereof, a therapeutically effective amount of a therapeutic agent that increases expression or activity of Ets2 polynucleotide or polypeptide, respectively. In one aspect, the cancer is a tumor. In another aspect, the cancer is stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma.

In another embodiment, the therapeutic agent is at least one polynucleotide located on human chromosome 21. In another aspect, the therapeutic agent is at least one polynucleotide located on the long arm of human chromosome 21. In one aspect, the therapeutic agent is at least one polynucleotide located on region 21q21-21q22.13 of human chromosome 21. In one aspect, the therapeutic agent is one or more polynucleotides located on region 21q21-21q22.13 of human chromosome 21. In one aspect, the therapeutic agent is selected from at least one of the genes indicated in Table 2. In another aspect, the therapeutic agent is the Ets2 polynucleotide. In yet another aspect, the therapeutic agent contains about 500,000 nucleotides on either side of the Ets2 polynucleotide.

In another embodiment of the present invention, a method is described that prevents or reduces the likelihood of developing cancer in a subject by administering to the subject a therapeutic agent that increases expression or activity of the Ets2 polynucleotide or polypeptide, respectively.

In another embodiment, the therapeutic agent is at least one polynucleotide located on human chromosome 21. In another aspect, the therapeutic agent is at least one polynucleotide located on the long arm of human chromosome 21. In one aspect, the therapeutic agent is at least one polynucleotide located on region 21q21-21q22.13 of human chromosome 21. In one aspect, the therapeutic agent is one or more polynucleotides located on region 21q21-21q22.13 of human chromosome 21. In one aspect, the therapeutic agent is selected from at least one of the genes indicated in Table 2. In another aspect, the therapeutic agent is the Ets2 polynucleotide. In yet another aspect, the therapeutic agent contains about 500,000 nucleotides on either side of the Ets2 polynucleotide.

In another embodiment of the present invention, a method is described of identifying a tumor repressor gene by measuring the level of expression of a polynucleotide suspected of encoding a tumor repressor or the activity of the tumor repressor in a sample, wherein an increased level of expression relative to a reference level from a cancerous sample is indicative that the polynucleotide is a tumor repressor. In one aspect, the polynucleotide encoding the tumor repressor or the activity of the tumor repressor is at least one polynucleotide located on human chromosome 21. In another aspect, the polynucleotide encoding the tumor repressor or the activity of the tumor repressor is at least one polynucleotide located on the long arm of human chromosome 21. In yet another aspect, the polynucleotide encoding the tumor repressor or the activity of the tumor repressor is at least one polynucleotide located on region 21q21-21q22.13 of human chromosome 21. In another aspect, the polynucleotide encoding the tumor repressor or the activity of the tumor repressor is one or more polynucleotides located on region 21q21-21q22.13 of human chromosome 21. In another aspect, the polynucleotide encoding the tumor repressor or the activity of the tumor repressor is selected from at least one of the genes indicated in Table 2.

In one aspect of the present invention, the sample is a biological sample. In another aspect, the sample is from the stomach, small intestine, large intestine, colon, peritoneum, lung, liver, breast, endometrial, ovary, testis, prostate, kidney, bladder, skin, brain, eye, mouth, pancreas, gall bladder, bone, endocrine or blood.

In one aspect, the subject is a human. In one aspect, the sample is from blood, saliva or urine.

### BRIEF DESCRIPTION OF THE DRAWINGS

FIG. 1 is a schematic showing comparative maps of aneuploid segments in mouse models. Numbers of Mmu16 genes conserved with Hsa21 and sizes in megabases (Mb) are shown.

FIG. 2 is a schematic showing the genotypes of the mouse crosses used in the methods of the invention.

FIG. 3 is a graph showing the distribution of tumor sizes for trisomic (open bars) and euploid (closed bars) mice. (a) Tumor number of mice at 120 days of age. (b) Tumor number of mice at 60 days of age. Arrows indicate mean tumor size in each genotype.

FIG. 4 is a graph showing the average tumor number at 120 days for the four genotypes, error bars indicate standard deviation. Number of mice analyzed, P value, and the gene copy number of Ets2 in each strain are indicated. *, statistical significance by Student's t-test of the designated pair.

FIG. 5 is a graph showing the average tumor number for each bi-monthly period.

FIG. 6 shows the levels of Ets2 RNA and protein. FIG. 6a is a graph showing relative expression of Ets2 RNA in the small intestine as determined by qPCR. Each open bar is the average from two mice, each of the two samples was run six times, error bars indicate standard error. FIG. 6b is a Western blot. Protein was extracted from MEFs of the indicated genotypes and analyzed by Western blotting. The ratio of Ets2 to tubulin was calculated for genotype pairs of samples. Independent lysates were assessed on a duplicate gel and the four measurements were averaged +/−standard deviation. FIG. 6c is a graph that shows relative Ets2 protein levels reflecting RNA levels and gene copy number in MEFs. The bar represents the mean for each group and the error bar designates standard deviation of each genotype (n=4 replicates per genotype except Ts65Tn where reliable measurements were obtained from three, not four samples). In all panels, Ets2 copy number is shown in parentheses.

### DETAILED DESCRIPTION OF THE INVENTION

Trisomy for chromosome 21 (Hsa21) is know to be the cause of Down syndrome (DS). This condition disrupts development of many tissues and the effects are generally deleterious to affected individuals. Among the phenotypes of DS, affected individuals have a greatly increased risk for childhood onset leukemia and for germ cell tumors. Paradoxically, several recent epidemiological studies provide evidence supporting a decades old suggestion that people with DS have a reduced incidence of solid tumors. These studies are limited by the small number of individuals with both DS and a specific form of cancer, resulting in inconsistent conclusions. Further, epidemiological studies cannot provide insight into the genetic mechanisms that result in this protection.

The present invention is based on the seminal discovery that individuals with DS have a lower incidence of solid tumors then do individuals without DS. Analysis of Ts65Dn, Apc^{Min} mice, which are trisomic for orthologs of about half of the genes on human chromosome 21, showed a significant reduction in tumor number and size compared to euploid mice. Thus, trisomy for genes found on Hsa21 is protective against tumorigenesis, and a small subset of these genes is sufficient to provide protection in a dosage sensitive manner. Stimulation of the same pathways affected when these genes are overexpressed provide prophylaxis against cancer.

Further studies demonstrated that the Ets2 gene contributed most of the dosage-sensitive effect on tumor number. The action of Ets2 as a repressor when it is overexpressed differs from tumor suppression, which requires normal gene function to prevent cellular transformation. An increase in Ets2 expression correlates with an increase in apoptosis and repression of tumor formation in mice models of DS. These results suggest that overexpression of one or more genes located on Hsa21 is able to repress cancer. Upregulation of Ets2 and, potentially, other genes involved in this kind of protective effect may provide a prophylactic effect in all individuals, regardless of ploidy.

Trisomy reveals a new category of cancer-related gene which is termed herein “tumor repressor.” These dosage sensitive genes reduce tumor incidence when slightly over-expressed and, in the invention described herein, increase incidence when expression levels are slightly reduced. Stimulation in euploid individuals of the same pathways affected by up-regulation of these tumor repressor genes in persons with DS may provide an approach for preventing cancer in all people.

It is possible that repression of tumorigenesis when Ets2 expression is elevated may in fact be a characteristic of many genes identified previously as oncogenes or tumor suppressor genes. Natural variation in average expression levels of ETS family (or other) repressor genes may exist in tumor-prone families without a known molecular basis for a high cancer frequency (reduced expression of Ets2) or in cancer-resistant families (elevated expression). Previous observations about the role of the Ets2 protooncogene in cancer could not have predicted that elevation of expression beyond euploid levels would provide a natural repression of tumor formation and growth.

Accordingly, the present invention provides methods of determining whether a subject has or is at risk of having cancer by measuring the level of expression of a polynucleotide encoding a tumor repressor or the activity of the tumor repressor in a sample from the subject, where a decrease in activity relative to a reference level is indicative of a subject that has or is at risk of having cancer. The polynucleotide encoding the tumor repressor or the activity of the tumor repressor can be located on human chromosome 21. The polynucleotide encoding the tumor repressor or the activity of the tumor repressor can be located on the long arm of human chromosome 21. Furthermore, the polynucleotide encoding the tumor repressor or the activity of the tumor repressor can be located on region 21q21-21q22.13 of human chromosome 21. The polynucleotide encoding the tumor repressor or the activity of the tumor repressor can be one or more polynucleotides located on region 21q21-21q22.13 of human chromosome 21. Additionally, the polynucleotide encoding the tumor repressor or the activity of the tumor repressor can be one or more of the genes indicated in Table 2.

The polynucleotide encoding the tumor repressor or the activity of the tumor repressor can also be the Ets2 polynucleotide. The polynucleotide encoding the tumor repressor or the activity of the tumor repressor can also be located about 500,000 nucleotides on either side of the Ets2 polynucleotide.

The sample can be a biological sample. The sample can be taken from the stomach, small intestine, large intestine, colon, peritoneum, lung, liver, breast, endometrial, ovary, testis, prostate, kidney, bladder, skin, brain, eye, mouth, pancreas, gall bladder, bone, endocrine saliva, urine, or blood.

As used herein, “cancer” refers to all types of cancers or neoplasm or malignant tumors and all types of cancers including solid tumors that are found in mammals. The cancer can be a tumor. The cancer can also be stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma. The subject of the present invention can be a human.

As used herein, the term “tumor repressor” means a dose sensitive gene which reduces tumor incidence when gene expression is elevated, as occurs for example when the gene is present in more then two gene copies. A tumor repressor gene which is over-expressed decreases tumor incidence, and increases tumor incidence when expression levels are reduced. As described herein, the tumor repressors of the present invention are polynucleotide derived from genes located on human chromosome 21.

The term “polynucleotide” or “nucleic acid sequence” refers to a polymeric form of nucleotides at least 10 bases in length. A polynucleotide may include a coding region with its associated regulatory sequences. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The polynucleotides of the invention can be ribonucleotides, deoxyribonucleotides, or modified forms of either nucleotide. The term includes single and double forms of DNA.

Polynucleotide sequences of the invention include DNA, cDNA and RNA sequences that encode a tumor repressor. It is understood that all polynucleotides encoding all or a portion of a tumor repressor can also be used in the invention methods, as long as they retain tumor repressor activity.

Such polynucleotides include naturally occurring, synthetic, and intentionally manipulated polynucleotides. For example, a polynucleotide of the present invention may be subjected to site-directed mutagenesis. The polynucleotide sequence for tumor repressors also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of the tumor repressor polypeptide encoded by the nucleotide sequence is functionally unchanged.

In general, a nucleic acid molecule that has “substantially the same nucleotide sequence” as a reference sequence will have greater than about 60% identity, such as greater than about 65%, 70%, 75% identity with the reference sequence, such as greater than about 80%, 85%, 90%, 95%, 97% or 99% identity to the reference sequence over the length of the two sequences being compared. Identity of any two nucleic acid sequences can be determined by those skilled in the art based, for example, on a BLAST 2.0 computer alignment, using default parameters.

Polynucleotide sequences used in the invention methods can be obtained by several methods. For example, the polynucleotides can be isolated using hybridization techniques that are well known in the art. These include, but are not limited to: 1) hybridization of genomic or cDNA libraries with probes to detect homologous nucleotide sequences and 2) antibody screening of expression libraries to detect cloned DNA fragments with shared structural features.

Specific polynucleotide sequences encoding a tumor repressor for use in the invention methods can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Since the present invention shows that a increased level of gene expression of a tumor repressor is associated with tumor repression, it is often desirable to directly determine whether a putative tumor repressor is overexpressed. Thus, the method may employ, for example, RNA quantification, including messenger RNA, in which the RNA can be quantitated by, for example, Northern blots. Additionally, enzymes, and/or conditions optimal for reverse transcribing the RNA template to DNA to perform an RT-PCR could be utilized. In addition, a DNA-RNA hybrid which contains one strand of each may be utilized. It is not necessary that the sequence to be amplified be present initially in a pure form; it may be a minor fraction of a complex mixture, such as contained in whole cell samples.

For purposes of the invention, a nucleic acid probe specific for the tumor repressor may be used to detect the presence of the polynucleotide or tumor repressor activity or in biological samples. Oligonucleotide primers based on any coding sequence region in the tumor repressor sequence are useful for amplifying DNA, for example by PCR. Any specimen containing a detectable amount of tumor repressor polynucleotide can be detected. Nucleic acid can also be analyzed by RNA in situ methods that are known to those of skill in the art. Preferred tissues for testing or treating according to the invention methods are tissue of stomach, small intestine, large intestine, colon, peritoneum, lung, liver, breast, endometrial, ovary, testis, prostate, kidney, bladder, skin, brain, eye, mouth, pancreas, gall bladder, bone, endocrine, saliva, urine, blood, and the like. Although the subject can be any mammal, preferably the subject is human.

Polynucleotide sequences encoding a tumor repressor can be expressed in vitro by DNA transfer into a suitable host cell, such as a tumor cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the polynucleotide encoding tumor repressor sequences may be inserted into a recombinant expression vector for expression either in vivo or in vitro. The term “recombinant expression vector” refers to a plasmid, virus or other vehicle known in the art that has been manipulated by insertion or incorporation of the tumor repressor genetic sequences. Such expression vectors contain a promoter sequence that facilitates the efficient transcription of the inserted genetic sequence of the host. The expression vector typically contains an origin of replication, a promoter, as well as specific genes that allow phenotypic selection of the transformed cells. Vectors suitable for use in the present invention include, but are not limited to the T7-based expression vector for expression in bacteria (Rosenberg, et al., Gene 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Polynucleotide sequences encoding tumor repressor sequences can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Methods that are well known to those skilled in the art can be used to construct expression vectors containing the tumor repressor sequences coding sequence and appropriate transcriptional/translational control signals. These methods include in vitro recombinant DNA techniques, synthetic techniques, and in vivo recombination/genetic techniques. See, for example, the techniques described in Maniatis, et al., 1989 Molecular Cloning A Laboratory Manual, Cold Spring Harbor Laboratory, N.Y.

Depending on the host/vector system utilized, any of a number of suitable transcription and translation elements, including constitutive and inducible promoters, transcription enhancer elements, transcription terminators, etc. may be used in the expression vector (see e.g., Bitter, et al., Methods in Enzymology 153:516-544, 1987). For example, when cloning in bacterial systems, inducible promoters such as pL of bacteriophage gamma, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the inserted tumor repressor coding sequence. In addition, the endogenous tumor repressor promoter, or a mutation thereof to protect the promoter from hypermethylation, may also be used to provide transcription machinery of the tumor repressor.

When the expression vector is introduced into a mammalian host cell in practice of the invention methods, a eukaryotic systems, and preferably mammalian expression systems, allows for proper post-translational modifications of expressed mammalian proteins to occur. Eukaryotic cells which possess the cellular machinery for proper processing of the primary transcript, glycosylation, phosphorylation, and advantageously, secretion of the gene product may be used as host cells for the expression of tumor repressor sequences.

Recombinant viruses or viral elements may be used to direct expression in mammalian cells. For example, when using adenovirus expression vectors, the tumor repressor coding sequence may be ligated to an adenovirus transcription/translation control complex, e.g., the late promoter and tripartite leader sequence. This chimeric gene may then be inserted in the adenovirus genome by in vitro or in vivo recombination.

Isolation and purification of microbial or host cell expressed polypeptide, or fragments thereof, provided by the invention, may be carried out by conventional means including preparative chromatography and affinity and immunological separations involving monoclonal or polyclonal antibodies.

The invention methods can utilize antibodies immunoreactive with polypeptides encoded by a tumor repressor, or immunoreactive fragments thereof. Antibody that consists essentially of pooled monoclonal antibodies with different epitopic specificities, as well as distinct monoclonal antibody preparations can be used. Monoclonal antibodies are made from antigen containing fragments of the protein by methods well known to those skilled in the art (Kohler, et al., Nature, 256:495, 1975). The term antibody as used in this invention is meant to include intact molecules as well as fragments thereof, such as Fab and F(ab′)₂, which are capable of binding an epitopic determinant on the tumor suppressor polypeptide.

Monoclonal antibodies can be used in the invention, for example, in immunoassays in which they can be utilized in liquid phase or bound to a solid phase carrier. In addition, the monoclonal antibodies in these immunoassays can be detectably labeled in various ways. Examples of types of immunoassays that can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays that are run in either the forward, reverse, or simultaneous modes, including immunohistochemical assays on physiological samples.

Those of skill in the art will know, or can readily discern, other immunoassay formats without undue experimentation. Additionally, those of skill will also appreciate that antibodies according to the present invention will be useful in other variations and forms of assays that are presently known or which may be developed in the future. These are intended to be included within the scope of the present invention.

The present invention also provides methods of treating a subject with cancer by administering to the subject in need thereof, a therapeutically effective amount of a therapeutic agent that increases expression or activity of Ets2 polynucleotide or polypeptide, respectively. In most individuals with DS for example, the Ets2 nucleotide sequence is present in three copies, and is overexpressed as compared to expression in a euploid cell, therefore, it is possible to design appropriate therapeutic or diagnostic techniques directed to this sequence. Thus, where a cancer is associated with the under expression of Ets2 associated with cancer, nucleic acid sequences that modulate Ets2 expression at the transcriptional or translational level can be used.

The cancer can be a tumor. The cancer can also be stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma.

The therapeutic agent can be at least one polynucleotide located on human chromosome 21. The therapeutic agent can be at least one polynucleotide located on the long arm of human chromosome 21. The therapeutic agent can be at least one polynucleotide located on region 21q21-21q22.13 of human chromosome 21. The therapeutic agent can be at least one or more polynucleotides located on region 21q21-21q22.13 of human chromosome 21. The therapeutic agent can be one or more of the genes indicated in Table 2. The therapeutic agent can be the Ets2 polynucleotide. The therapeutic agent can be about 500,000 nucleotides on either side of the Ets2 polynucleotide.

Generally, the terms “treating,” “treatment” and the like are used herein to mean affecting a subject, tissue or cell to obtain a desired pharmacologic and/or physiologic effect. The effect may be prophylactic in terms of completely or partially preventing cancer, and/or may be therapeutic in terms of a partial or complete cure for cancer and/or adverse effect attributable to cancer. “Treating” as used herein covers any treatment of, or prevention of, or inhibition of cancer in a subject. The subject can be an invertebrate, a vertebrate, or a mammal, and particularly a human.

As used herein, the term “therapeutically effective” amount refers to the quantity of a component which is sufficient to yield a desired therapeutic response without undue adverse side effects (such as toxicity, irritation, or allergic response) commensurate with a reasonable benefit/risk ratio when used in the manner of this invention. The specific “safe and effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives.

In one aspect of the invention, the therapeutic agent is the Ets2 polynucleotide. As used herein, the term “Ets2 polynucleotide” refers to a polynucleotide that possesses a biological function or activity which is identified through a defined functional assay and which is associated with a particular biologic, morphologic, or phenotypic alteration in the cell. Functional fragments of the Ets2 polynucleotide include fragments of Ets2 that retain the activity of e.g., tumor repressor activity of Ets2. Smaller polynucleotides containing the biological activity of Ets2 are included in the invention. The biological function, for example, can vary from a polynucleotide fragment which encodes a peptide as small as an epitope to which an antibody molecule can bind, to a large polynucleotide containing the Ets2 polynucleotide and 500,000 nucleotides on either side of the Ets2 polynucleotide.

Minor modifications of the polynucleotide sequence may result in polynucleotides that have substantially equivalent activity as compared to the Ets2 polynucleotide described herein. Such modifications may be deliberate, as by site-directed mutagenesis, or may be spontaneous. All of the polynucleotide produced by these modifications are included herein as long as the tumor repressor activity of the polynucleotide is present. Further, deletion of one or more nucleic acids can also result in a modification of the structure of the resultant molecule without significantly altering its activity. This can lead to the development of a smaller active molecule that would have broader utility. For example, it possible to remove terminal or non-coding nucleotides that may not be required for Ets2 activity.

The present invention contemplates therapeutic agents useful for practicing the therapeutic methods described herein. Therapeutic agents of the present invention contain a physiologically tolerable carrier together with at least one species of a therapeutic agent that increases expression or activity of the Ets2 polynucleotide or polypeptide described herein, dissolved or dispersed therein as an active ingredient. The therapeutic agent can also increase the genes or gene products located downstream of Ets2. In a preferred embodiment, the therapeutic composition is not immunogenic when administered to a human patient for therapeutic purposes.

The preparation of a therapeutic agent that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified. Thus, a therapeutic agent containing composition can take the form of solutions, suspensions, tablets, capsules, sustained release formulations or powders, or other compositional forms.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic agent of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol, and other solutes.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

The present invention also provides gene therapy for the treatment of cell proliferative disorders. Such therapy would achieve its therapeutic effect by introduction of the polynucleotide encoding a tumor repressor, or fragments thereof, into cells having cancer. Delivery of such polynucleotides can be achieved using a recombinant expression vector such as a chimeric virus or a colloidal dispersion system. Colloidal dispersion systems include macromolecule complexes, nanocapsules, microspheres, beads, and lipid-based systems including oil-in-water emulsions, micelles, mixed micelles, and liposomes. RNA, DNA and intact virions can be encapsulated within the aqueous interior and be delivered to cells in a biologically active form (Fraley, et al., Trends Biochem. Sci., 6:77, 1981).

Various viral vectors which can be utilized for gene therapy as taught herein include adenovirus, herpes virus, vaccinia, or, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a tumor repressor sequence of interest into the viral vector, along with another gene which encodes the ligand for a receptor on a specific target cell, for example, the vector is now target specific. Retroviral vectors can be made target specific by attaching, for example, a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome or attached to a viral envelope to allow target specific delivery of the retroviral vector containing the polynucleotide.

The present invention also provides methods of preventing or reducing the likelihood of developing cancer in a subject by administering to the subject a therapeutic agent that increases expression or activity of the Ets2 polynucleotide or polypeptide, respectively.

The present invention also provides methods of identifying a tumor repressor gene by measuring the level of expression of a polynucleotide suspected of encoding a tumor repressor or the activity of the tumor repressor in a sample, wherein a increased level of expression relative to a reference level from a cancerous sample is indicative that the polynucleotide is a tumor repressor. The polynucleotide encoding the tumor repressor can be a polynucleotide located on human chromosome 21. The polynucleotide can be at least one polynucleotide located on the long arm of human chromosome 21. The polynucleotide can be at least one polynucleotide located on region 21q21-21q22.13 of human chromosome 21. The polynucleotide can be at least one or more polynucleotides located on region 21q21-21q22.13 of human chromosome 21. The polynucleotide can be one or more of the genes indicated in Table 2. The polynucleotide can be the Ets2 polynucleotide. The polynucleotide can be about 500,000 nucleotides on either side of the Ets2 polynucleotide.

It should be noted that as used herein and in the appended claims, the singular forms “a,” “and,” and “the” include plural referents unless the context clearly dictates otherwise. Thus, for example, reference to “a cell” includes a plurality of such cells and reference to “the restriction enzyme” includes reference to one or more restriction enzymes and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the methodologies which are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following example is intended to illustrate but not to limit the invention in any manner, shape, or form, either explicitly or implicitly. While they are typical of those that might be used, other procedures, methodologies, or techniques known to those skilled in the art may alternatively be used.

### EXAMPLES

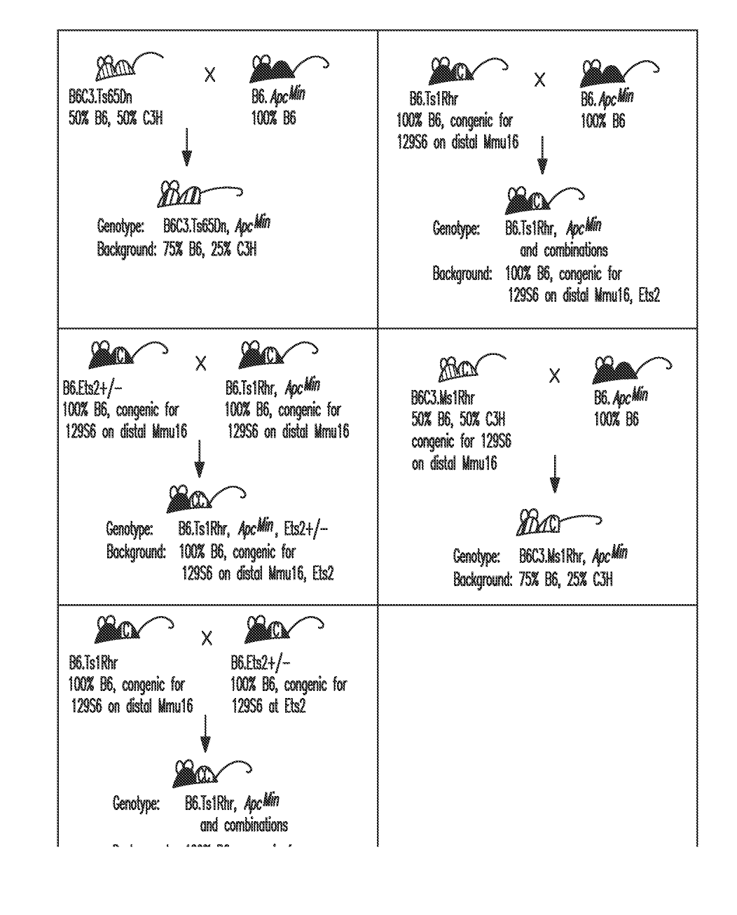

Mice. C57BL/6J-Apc^{Min} mice (herein Apc^{min}) were purchased from the Jackson Laboratory (Bar Harbor, Me.) and maintained by repeated backcrossing to C57B1/6J (B6) mice. B6EiC3Sn α/A-Ts(17¹⁶)65Dn (herein Ts65Dn) mice were purchased from the Jackson Laboratory. B6C.3Del(16Cbrl-ORF9)1Rhr (herein Ms1Rhr) and Ts65Dn mice were maintained as an advanced intercross by crossing to (B63C3H/HeJ)F1 mice. B6.Dup(Cbrl-ORF9)1Rhr mice (herein Ts1 Rhr) were backcrossed eight or more generations onto C57B1/6J. Mice carrying a null allele of Ets2 (herein Ets2+/− mice) were backcrossed for more than nine generations onto B6 before being used in these experiments. The genetic backgrounds of all mice produced for this study are shown in FIG. 2. In general, groups of euploid and trisomic littermates from related mothers were used in crosses that generated aneuploid mice to minimize genetic variation.

Genotyping. Apc^{Min} mice were genotyped by mutation-specific PCR, as described by the Jackson Laboratory. Ts1Rhr and Ms1Rhr mice were genotyped by PCR. For Ts1Rhr, primers were used for the wild-type Cbrl gene (CBR 20F-CTGCTCCTTCTTCTGGGCTT (SEQ ID. NO.:1), CBR 20R-CTCACGGTCATCTGGCTTCT) (SEQ ID. NO.:2) and the targeted Cbrl gene, (HYG 19F-CCGTCAGGACATTGTTGGA (SEQ ID. NO.:3), HYG 19R-CCGTAACCTCTGCCGTTCA) (SEQ ID. NO.:4), using the following cycling conditions: 94° C. 2 min, (94° C., 30 sec, 55° C. 1 min, 72° C. 45 sec)×30, 72° C. 7 min. MsIRhr mice were typed using primers for the wild-type Cbrl gene and the targeted Cbrl gene (MXP F2-GGACGGTTGGAGAAGAAGGT (SEQ ID. NO.:5), CBRH R2-CTCGTCCTGCAGTTCATTCA) (SEQ ID. NO.:6). Ts65Dn mice were genotyped by fluorescent in situ hybridization (FISH).

The Momls gene was genotyped by mutation specific quantitative PCR, and analyzed on a LightCycler (Roche Diagnostics Corporation, Manheim, Germany). Primers were designed to amplify the wild-type (Mom Common-TGGGGAAATGATTTGGCTTA (SEQ ID. NO.:7), Mom WT-TGGCATCCTTGGGGGAT) (SEQ ID. NO.:8) and mutant (Mom Common, Mom MUT-TGGCATCCTTGGGGGAA) (SEQ ID. NO.:9) alleles. These primers were used with the LightCycler FastStart DNA Master SYBR Green I kit (Roche Diagnostics Corporation, Manheim, Germany), using the following cycling conditions: 95° C. 10 min, (95° C. 10 sec, 58° C. 5 sec, 72° C. 20 sec)×55, followed by a melting curve analysis. An absent allele caused a five-cycle delay in amplification, and a coordinate shift in DNA melting temperature.

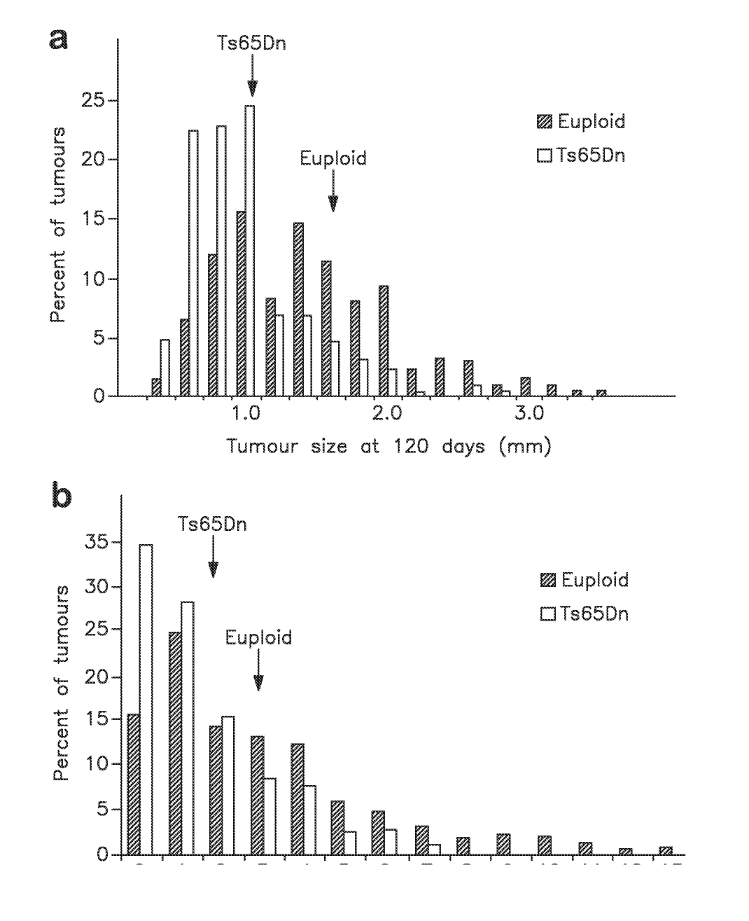

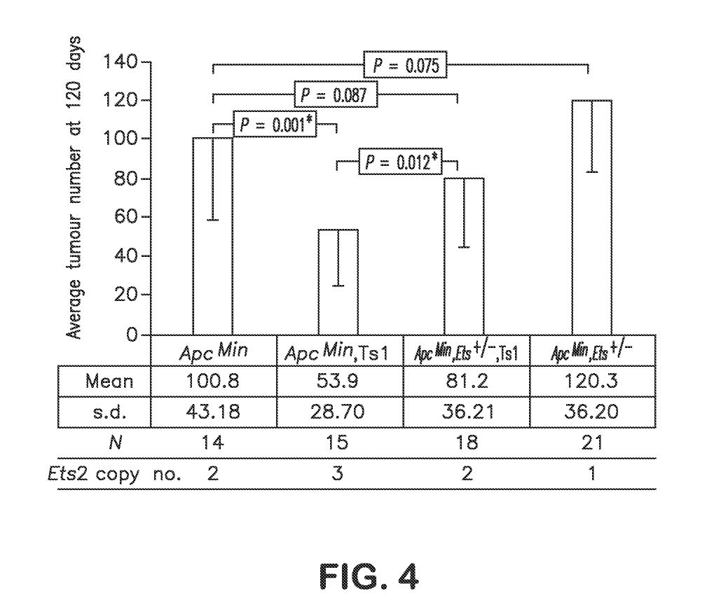

Tumor Analysis. All animals were assessed blind to genotype in all assays. Groups of littermate mice from closely related mothers (and inbred fathers) were euthanized at 12062 days of age. Intestines were removed and rinsed, then cut longitudinally and placed in fresh PBS. Tumors were counted under 20× magnification across the entire length of the small intestine. For Table 3, tumors were scored if they were <0.4 mm in diameter; small tumors that did not involve multiple crypts were excluded. Independent crosses were assessed by independent observers to confirm the effects of aneuploidy on tumorigenesis. The Ts65Dn Apc^{Min} tumor analysis was done three times by two observers (data in Table 3, FIG. 3a and FIG. 3b) and the Ts1Rhr 3 Apc^{Min} analysis was performed twice by two observers (Table 3 and FIG. 4). A summary of the crosses and data collection process is in FIG. 2.

For visible tumors (at 20× magnification), tumor size was determined for the longest axis of the tumor using an eyepiece reticule. Statistical significance was determined using a Student's t-test. For microscopic tumors, intestines were recovered from Apc^{Min} and Ts65Dn, Apc^{Min} littermates 60 days of age. Intestines were removed, washed in PBS several times and then cut into three sections (proximal to distal). Each section was cut open longitudinally and fixed overnight in 10% formalin. The next day the intestine was rolled up and embedded in paraffin as a ‘Swiss roll,’ and ten slides each containing 3 sections of 6 microns thick were recovered at an interval of 50 microns. Slides were deparaffinized, stained with b-catenin antibody (BD Biosciences Clone 14, Vector M.O.M immunodetection Kit) and co-stained with haematoxylin, and tumors from ten slides per mouse were measured under a light microscope with an eye piece reticule. Tumor size and number were counted and results compiled.

RNA and protein analysis. Mouse embryo fibroblasts (MEFs) were established from fetuses at E13.5. Fetuses were removed and the visceral tissue separated. Remaining tissue was minced in Trypsin/EDTA and incubated at 37° C. for an hour. Trypsin was neutralized by addition of medium (DMEM plus 10% serum and antibiotics) and cells collected and plated, taking care to avoid transfer of larger pieces of tissue. The next day, cells were re-fed, then passaged as they reached confluence. For these experiments, cells were used between 6-8 passages.

Total RNA was isolated from mouse small intestine or MEFs with TRIzol reagent (Invitrogen) and RNeasy Mini Kit (Qiagen), including a DNase I treatment step. RNA concentration was determined by UV spectrophotometry and 1 mg was reverse transcribed with GeneAmp RNA PCR kit (Applied Biosystems). After dilution, 10 ng of complementary DNA was amplified by real-time PCR with SYBR Green PCR master mix (Applied Biosystems) using specific primers for Ets2 (Forward, AGAGAAGGGAGCACAGCAAA (SEQ ID. NO.:10); Reverse, AAGAACATGGACCAAGTGGC) (SEQ ID. NO.:11) and b-actin (Forward, AGTGTGACGTTGACATCCGTA (SEQ ID. NO.:12); Reverse, CCAGAGCAGTAATCTCCTTCT) (SEQ ID. NO.:13). Real-time PCR was carried out under the following conditions: 10 min at 95° C., followed by 40 cycles of 15 s at 95° C. and 1 min at 60° C. (Applied Biosystems 7500 System). Ct values were determined by subtracting the average b-actin Ct value from the average Ets2 Ct value. The standard deviation of the difference was calculated from the standard deviation of Ets2 and b-actin values. After each real-time RT-PCR, a melting profile was done to rule out non-specific contributions from PCR products and primer dimers.

For Western blots, whole cell lysates were prepared by lysing MEFs with RIPA buffer, and 100 mg of protein from each sample was separated by 8% SDS PAGE. The membrane was blotted overnight with anti-Ets2 1:1,000, 5% milk in 0.05% TBST (TBST is 0.05% Tween-20, 20 mM Tris-HCl pH7.6 and 150 mM NaCl), probed with anti-Rabbit HRPand developed for ECL. Blots were stripped and reprobed with anti-tubulin (1:1,000 in 5% Milk in 0.05% TBST) antibody. Scanned images of each blot were inverted by NIH Image J and the density calculated for Ets2 and tubulin in each sample. The background was measured and subtracted and the ratio of Ets2/tubulin density was used to compare protein expression level of Ets2 in MEFs of different genotypes. The average level of Ets2:tubulin in euploid mice was arbitrarily set at 1.0 and Ets2 levels in other genotypes were calculated in proportion.

Ts65Dn, ApcMin Mice Have Reduced Tumor Incidence. Female Ts65Dn mice were crossed to Apc^{Min} males, and the number of tumors in the small intestine was determined in Ts65Dn, Apc^{Min} progeny at 120 days of age. Trisomic mice showed a significant 44% reduction in the number of tumors compared to their euploid, Apc^{Min} littermates, from 45.4 to 25.3 tumors (Table 3). This establishes a biological basis for the effects of trisomy on tumor formation and shows that trisomy for orthologues of about half of the genes on Hsa21 is sufficient to reduce tumor incidence in this model.

TABLE 3
Average numbers of intestinal tumors in aneuploid
and euploid mice at 120 days of age

| | | | | t-test |
|---|---|---|---|---|
| | Average no. | | No. of | significance |
| | of tumors | s.d. | mice | (P value) |
| Either Mom1 allele | | | | |
| Euploid | 45.4 | 29.9 | 24 | 0.008 |
| Ts65Dn | 23.8 | 14.2 | 10 | |
| Mom1^{s/s} | | | | |
| Euploid | 62.6 | 26.4 | 14 | 0.0028 |
| Ts65Dn | 31.2 | 13.7 | 6 | |
| Mom1^{s/r} | | | | |
| Euploid | 21.3 | 13.3 | 10 | 0.105 |
| Ts65Dn | 12.8 | 5.0 | 4 | |
| Segmental aneuploidies* | | | | |
| Euploid (B6) | 107.3 | 45.0 | 16 | 0.043 |
| Ts1Rhr(B6) | 79.6 | 29.9 | 21 | 0.048 |
| Euploid (B6/C3H) | 37.0 | 16.0 | 9 | |
| Ms1Rhr(B6/C3H) | 74.4 | 39.7 | 7 | |

*Genetic background is shown in parenthesis. Ts65Dn and euploid controls are B6/C3H.

The Apc^{Min} mutation is inbred onto B6, and these mice are therefore homozygous for the permissive (high tumor incidence) mutant allele of Momls. C3H mice carry a wild-type Momlr allele, which reduces the number of tumors in Apc^{Min} mice. Ts65Dn mice were maintained as a B6C3 advanced intercross and thus the progeny of this cross can be Momls/Momls, s/r or r/r at this locus. The tumor number in euploid and Ts65Dn mice carrying the Apc^{Min} mutation with respect to the genotype at Mom1 were analyzed. The data was reanalyzed data considering the inheritance of susceptible or resistant alleles of the modifier of Min 1 (Mom1) locus that result in higher or lower tumor number (Momls and Momlr, respectively; genetic background of all crosses is shown in FIG. 2) The inheritance of a single Momlr allele reduced the average tumor number from 62.6 to 21.3 in euploid mice (66%) as expected, and a similar 59% reduction occurred in Ts65Dn (Table 3). Trisomic, Momls/Momls mice had a highly significant 50% reduction in small intestine tumor number compared to euploid, Moml/Moml mice (Table 3). The presence of a wild type allele resulted in substantially fewer tumors in both trisomic and euploid Momls/r animals compared to their Momls/Momls littermates. Trisomic mice that inherited a Momlr allele (Momls/r) also had substantially reduced tumor numbers relative to euploid mice, although this reduction did not reach a statistically significant level in the small sample of Ts65Dn, Momls/r mice available for this post-hoc analysis. Thus, the Momlr effect seems to be additive with the protective effect of trisomy, suggesting that independent mechanisms are involved.

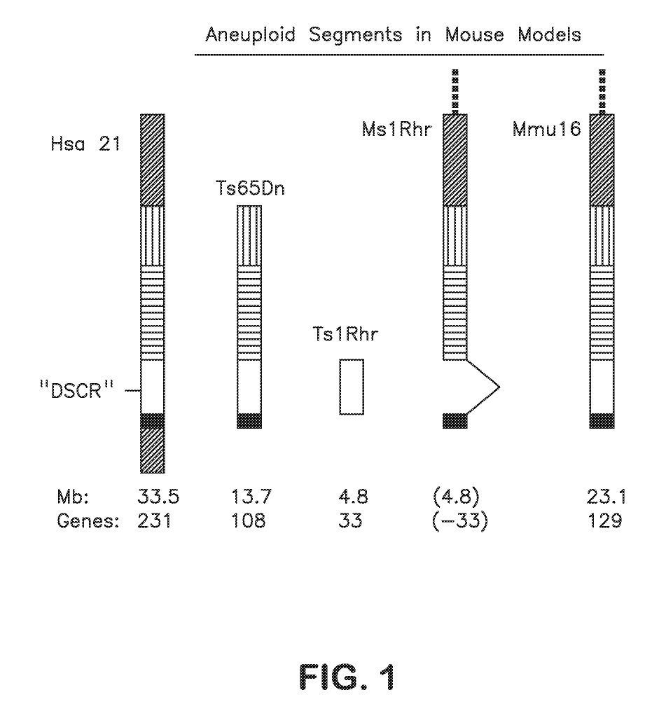

Tumor Incidence in Ts1Rhr and Ms1Rhr mice carrying ApcMin. Ts1Rhr mice were analyzed in order to narrow the candidate region for the gene or genes responsible for reduced tumor number. These mice have segmental trisomy for 33 of the genes that are in triplicate in Ts65Dn (FIG. 1). These genes represent a “critical region” of Hsa21, previously thought to be sufficient to cause several phenotypes of Down's syndrome. Ts1Rhr, Apc^{Min} mice had a significant 26% reduction in the average number of tumors in the small intestine when compared to euploid, Apc^{Min} mice (Table 3).

When Apc^{Min} mice were crossed to Ms1Rhr, which have segmental monosomy for the 33 genes that are triplicated in Ts1Rhr, a significant 101% increase was observed in tumor number in the monosomic mice compared to euploid mice (Table 3). These results demonstrate that a gene (or combination of genes) in this region is dosage sensitive in both directions with respect to the effect on tumor number.

Tumor size is decreased in Ts65Dn but not Ts1Rhr or Ms1Rhr mice. The size of tumors in a given genetic background provides one indicator of tumor initiation and growth rates. The size of tumors between trisomic and euploid Apc^{Min} mice were compared (FIG. 3a). Ts65Dn, Momls/s mice showed a significant 34% reduction in average and median tumor size at 120 days compared to euploid (P=0.005). Note that Ts65Dn mice in this experiment had 48% fewer tumors than did euploid animals, a significantly lower level that replicates in this independent cross the reduction in tumor number reported for the independent cohort of mice represented in Table 3 (P=0.04, N54 euploid, 5 Ts65Dn).

To determine whether this difference was evident earlier in the course of tumor formation, intestines of trisomic and euploid mice carrying the Apc^{Min} allele were immunostained for b-catenin at 60 days of age. As at 120 days of age, the number and average size of tumors in Ts65Dn mice was significantly less than in their euploid counterparts (FIG. 3b). Microtumors varied in size, sometimes restricted to a single crypt or involving adjacent transformed crypts. Untransformed crypts show b-catenin accumulated on membranes while tumors show high levels of nuclear and cytoplasmic staining. No tumors were seen at 30 days of age in two euploid or one trisomic Apc^{Min} mouse after b-catenin staining. Thus the repression of tumor number and size in Ts65Dn mice was evident early in tumor formation.

In contrast to Ts65Dn mice, tumor size was not different from euploid in either Ts1Rhr or Ms1Rhr mice (data not shown). The absence of a tumor size phenotype even though tumor number is reduced in Ts1Rhr mice indicates that multiple genes on Mmu16 (and Hsa21) may contribute to different aspects of tumor repression caused by trisomy.

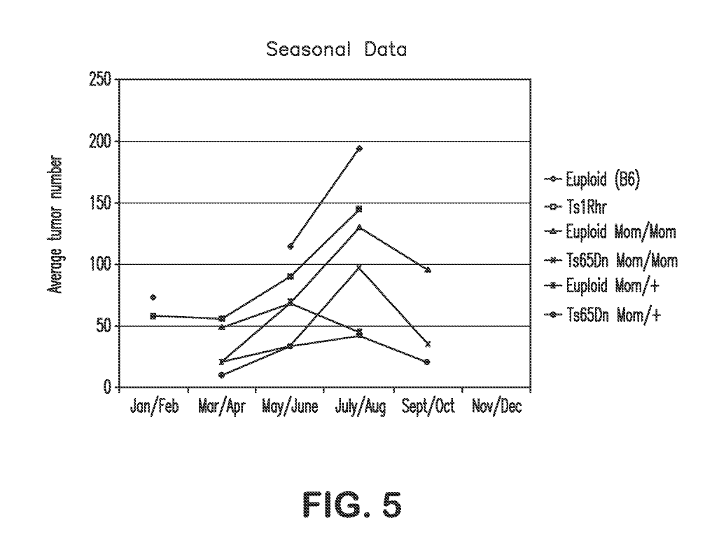

APC Mice Exhibit Seasonal Variation In Tumor Number. A pronounced seasonal variation in tumor number in Apc^{Min} mice was observed, regardless of ploidy. The highest numbers occurred in mice analyzed during the summer months (FIG. 5). This two- to four-fold seasonal variation was observed in mice inbred onto C57BL/6 in the Ts1Rhr experiments, as well as with mice on a mixed B6×3H background in the Ts65Dn and Ms1Rhr experiments. Differences in tumor number reported here are based on all observations. The significance of differences due to trisomy was greatly increased during the summer months.

Ets2 is a Tumor Repressor Gene. The 33 genes at dosage imbalance in Ts1Rhr and Ms1Rhr mice include several possible candidates for the tumor number effect (Table 2), including the Ets2 “proto-oncogene.” Although generally considered a “pro-cancer” gene, Ets2 has several activities consistent with a role in repressing the early stages of transformation. A three-way cross was preformed to produce mice carrying Apc^{Min} that were either euploid or had the Ts1Rhr segmental trisomy, and which segregated an allele of Ets2 that deletes exons 3-5 and fails to produce functional Ets2 protein. Tumors were counted at 120 days (FIG. 4). This independent cohort of mice replicated the observation (Table 3) that trisomy for three copies of Ets2 and 32 flanking genes in Ts1Rhr results in a significantly reduced tumor incidence, from a mean of 100.8 to 53.9 (P=0.001). However, when Ets2 was returned to the normal two copy level in mice that were still trisomic for the 32 flanking genes (Apc^{Min}, Ets21/2, Ts1Rhr), average tumor number increased significantly to 81.2 (P=0.012). Thus, a substantial portion, though not all, of the tumor repression in Ts1Rhr is accounted for by the extra copy of Ets2.

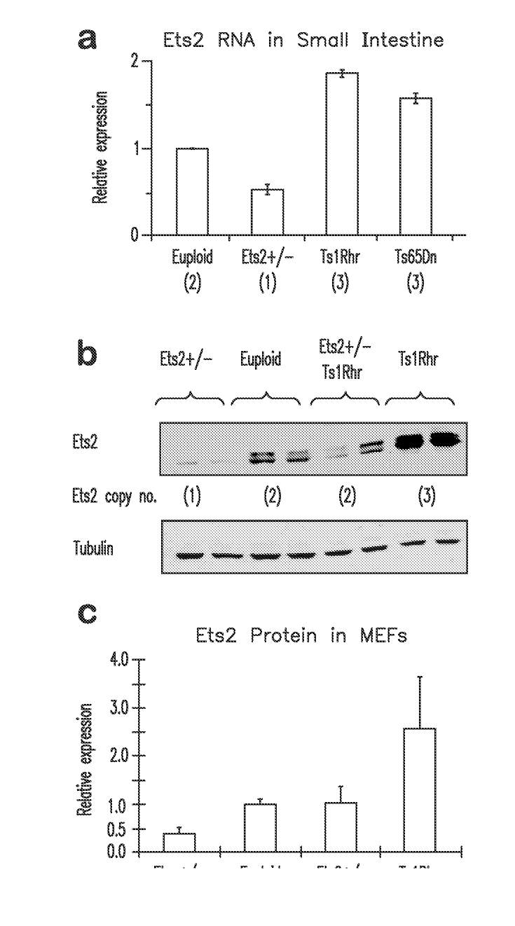

Mice that carried a single copy of Ets2 in a euploid background showed a substantial, 20% increase in tumor frequency (P=0.075), reminiscent of the increase in tumors in Ms1Rhr mice, which carry a single copy of this gene. These mice developed severe disease much earlier than mice of other genotypes and several did not survive long enough for tumors to be counted. Thus this difference in tumor number is probably under-represented. Ets2 messenger RNA and protein levels corresponded directly to gene copy number in all of the genotypes (FIG. 6).

The advanced backcross generations of Ts1Rhr (zN8) and Ets2+/−(zN9) onto B6 assure minimal background variation in these strains. Both of these and the genetically engineered segment in MslRhr were derived in ES cells from the 129S6 strain and will retain this background as a congenic segment in the vicinity of the engineered regions. The fact that Ts65Dn, which has never had any 129S6 strain contribution, has the same repressor phenomenon as TslRhr argues against an exclusive effect of a tightly linked 129S6-derived tumor number modifier in TslRhr. Further, Ets2+/− mice, congenic for the region surrounding Ets2, have more tumors while Ts1Rhr mice, which are congenic for a region that includes Ets2, have less. A 129S6-derived repressor flanking the Ts1Rhr site is unlikely because similar flanking congenic regions from 129S6 occur in Ts1Rhr, which shows fewer tumors, and in Ms1Rhr, which has more tumors. Finally, extensive studies have mapped more than a dozen loci that modify tumor number in Apc^{Min} mice, but no modifier of Min has been mapped in any strain to mouse chromosome 16 and no tumor suppressor activity maps to distal mouse chromosome 16.

Mouse models of DS were utilized to determine the rates of Apc^{Min}-induced tumorigenesis. The results described above show that Ts65Dn mice developed significantly fewer intestinal tumors than their euploid littermates. This demonstrates that trisomy for approximately half of the Hsa21 orthologs is capable of reducing tumorigenesis. This reduction in Apc^{Min}-induced tumorigenesis demonstrates that the differences in tumor incidence/mortality, observed in the epidemiological studies, is not solely a result of environmental factors, such as exposure to cigarette smoke, alcohol, and UV radiation. Instead, this reveals that there is an underlying genetic component associated with the reduced risk of tumorigenesis among individuals with DS. Furthermore, tumor reduction in Ts65Dn mice demonstrates that only half the Hsa21 orthologs are required for this protective phenotype. Moreover, trisomy reduces tumor number to nearly the same extent as the potent modifier gene, Moml, does.

The results also showed that the size of Apc^{Min}-tumors was reduced in Ts65Dn mice. This suggests that trisomy results in slower tumor induction and/or delayed onset of tumorigenesis, rather than a reduction in the number of cells initiating transformation. Compared to euploid mice, Ts65Dn mice had the same number of mitotic cells, but exhibited a four-fold increase in the number of apoptotic cells. Ts65Dn has 104 genes that have additional effects on tumor growth.

Ts1Rhr mice, which are trisomic for approximately 33 Hsa21 orthologs, had a significant reduction in tumor number compared to euploid littermates. However, Ts1Rhr mice did not protect to the same extent as Ts65Dn mice did. Furthermore, unlike Ts65Dn mice, tumors from Ts1Rhr mice were not smaller than euploid tumors. These results indicate that the degree of protection may be somewhat smaller in Ts1Rhr than in Ts65Dn. Nonetheless, it is clear that this small subset of Hsa21 orthologs is sufficient to provide a significant level of protection against Apc^{Min}-induced tumors.

The results also showed that monosomic Ms1Rhr mice exhibited a significant increase in tumor burden. This supports the notion that gene(s) within the Ts1Rhr trisomic region modulate tumor number in a dosage dependent manner. This dosage dependence seen in Ts1Rhr and Ms1Rhr mice indicates that trisomy of the 5 Mb region is protective, while monosomy of this region predisposes to greater tumor numbers.

Additionally, a fluctuation in tumor number throughout the year occurred. This fluctuation was observed over a two-year period, and was seen in all the genotypes studied. In humans, there are several environmental risk factors for colon cancer, including red meat consumption, smoking, and alcohol consumption. Protective factors include non-steroidal anti-inflammatory drugs, vegetable consumption, calcium, folate, hormone replacement therapy, and physical exercise. In Apc^{Min} mice, many factors, including NSAIDs, DNA methyltransferase activity, vitamin D, caloric intake, indole 3-carbanol (in cruciferous vegetables), n-3 fatty acids, bile acid, high fat, physical exercise, and soy isoflavones have been shown to modulate tumor number. It is likely that seasonal fluctuations in diet, and/or hormone levels played a role in this variability.

Although the invention has been described with reference to the above example, it will be understood that modifications and variations are encompassed within the spirit and scope of the invention. Accordingly, the invention is limited only by the following claims.

1. A method of determining whether a subject has or is at risk of having cancer comprising measuring the level of expression of a polynucleotide encoding a tumor repressor or the activity of the tumor repressor in a sample from the subject, wherein a decreased level relative to a reference level is indicative of a subject that has or is at risk of having cancer.

2. The method of claim 1, wherein the polynucleotide encoding the tumor repressor or the activity of the tumor repressor comprises at least one polynucleotide located on human chromosome 21.

3. The method of claim 1, wherein the polynucleotide encoding the tumor repressor or the activity of the tumor repressor comprises at least one polynucleotide located on the long arm of human chromosome 21.

4. The method of claim 1, wherein the polynucleotide encoding the tumor repressor or the activity of the tumor repressor comprises at least one polynucleotide located on region 21q21-21q22.13 of human chromosome 21.

5. The method of claim 1, wherein the polynucleotide encoding the tumor repressor or the activity of the tumor repressor comprises one or more polynucleotides located on region 21q21-21q22.13 of human chromosome 21.

6. The method of claim 2, wherein the polynucleotide encoding the tumor repressor or the activity of the tumor repressor are selected from at least one of the genes indicated in Table 2.

7. The method of claim 1, wherein the polynucleotide encoding the tumor repressor or the activity of the tumor repressor comprises the Ets2 polynucleotide.

8. The method of claim 7, wherein the polynucleotide encoding the tumor repressor or the activity of the tumor repressor further comprises about 500,000 nucleotides on either side of the Ets2 polynucleotide.

9. The method of claim 1, wherein the sample is a biological sample.

10. The method of claim 1, wherein the sample is from the stomach, small intestine, large intestine, colon, peritoneum, lung, liver, breast, endometrial, ovary, testis, prostate, kidney, bladder, skin, brain, eye, mouth, pancreas, gall bladder, bone, endocrine or blood.

11. The method of claim 1, wherein the cancer is a tumor.

12. The method of claim 1, wherein the cancer is stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma.

13. A method of treating cancer in a subject comprising administering to the subject in need thereof, a therapeutically effective amount of a therapeutic agent that increases expression or activity of Ets2 polynucleotide or polypeptide, respectively.

14. The method of claim 13, wherein the cancer is a tumor.

15. The method of claim 13, wherein the cancer is stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma.

16. The method of claim 13, wherein the therapeutic agent comprises at least one polynucleotide located on human chromosome 21.

17. The method of claim 13, wherein the therapeutic agent is selected from at least one of the genes indicated in Table 2.

18. The method of claim 13, wherein the therapeutic agent comprises the Ets2 polynucleotide.

19. A method of preventing or reducing the likelihood of developing cancer in a subject comprising administering to the subject a therapeutic agent that increases expression or activity of the Ets2 polynucleotide or polypeptide, respectively.

20. the method of claim 19, wherein the cancer is stomach cancer, kidney cancer, bone cancer, liver cancer, brain cancer, skin cancer, oral cancer, lung cancer, pancreatic cancer, colon cancer, intestinal cancer, myeloid leukemia, melanoma, glioma, thyroid follicular cancer, bladder carcinoma, myelodysplastic syndrome, breast cancer, low grade astrocytoma, astrocytoma, glioblastoma, medulloblastoma, renal cancer, prostate cancer, endometrial cancer or neuroblastoma.

21. The method of claim 19, wherein the therapeutic agent comprises at least one polynucleotide located on human chromosome 21.

22. The method of claim 19, wherein the therapeutic agent is selected from a least one of the genes indicated in Table 2.

23. The method of claim 19, wherein the therapeutic agent comprises the Ets2 polynucleotide.

24. A method of identifying a tumor repressor gene comprising measuring the level of expression of a polynucleotide suspected of encoding a tumor repressor or the activity of the tumor repressor in a sample, wherein an increased level of expression relative to a reference level from a cancerous sample is indicative that the polynucleotide is a tumor repressor.

25. The method of claim 24 wherein the polynucleotide encoding the tumor repressor comprises a polynucleotide located on human chromosome 21.

26. The method of claim 24, wherein the polynucleotide encoding the tumor repressor is selected from at least one of the genes indicated in Table 2.

27. The method of claim 24, wherein the sample is a biological sample.

28. The method of claim 24, wherein the sample is derived from a tissue selected from the group consisting of stomach, small intestine, large intestine, colon, peritoneum, lung, liver, breast, endometrial, ovary, testis, prostate, kidney, bladder, skin, brain, eye, mouth, pancreas, gall bladder, bone, endocrine or blood.

29. The method of claim 1, wherein the subject is a human.

30. The method of claim 1, wherein the sample is from blood, saliva or urine.


SEQUENCE LISTING (13 sequences)

SEQ ID NO: 1 - DNA, 20 bp, Artificial sequence
  Primer
ctgctccttcttctgggctt

SEQ ID NO: 2 - DNA, 20 bp, Artificial sequence
  Primer
ctcacggtcatctggcttct

SEQ ID NO: 3 - DNA, 19 bp, Artificial sequence
  Primer
ccgtcaggacattgttgga

SEQ ID NO: 4 - DNA, 19 bp, Artificial sequence
  Primer
ccgtaacctctgccgttca

SEQ ID NO: 5 - DNA, 20 bp, Artificial sequence
  Primer
ggacggttggagaagaaggt

SEQ ID NO: 6 - DNA, 20 bp, Artificial sequence
  Primer
ctcgtcctgcagttcattca

SEQ ID NO: 7 - DNA, 20 bp, Artificial sequence
  Primer
tggggaaatgatttggctta

SEQ ID NO: 8 - DNA, 17 bp, Artificial sequence
  Primer
tggcatccttgggggat

SEQ ID NO: 9 - DNA, 17 bp, Artificial sequence
  Primer
tggcatccttgggggaa

SEQ ID NO: 10 - DNA, 20 bp, Artificial sequence
  Primer
agagaagggagcacagcaaa

SEQ ID NO: 11 - DNA, 20 bp, Artificial sequence
  Primer
aagaacatggaccaagtggc

SEQ ID NO: 12 - DNA, 21 bp, Artificial sequence
  Primer
agtgtgacgttgacatccgta

SEQ ID NO: 13 - DNA, 21 bp, Artificial sequence
  Primer
ccagagcagtaatctccttct